(19) United States
(12) Patent Application Publication
(10) Pub. No.: US 2008/0239920 A1
(43) Pub. Date: Oct. 2, 2008

(54) Wireless transmission of temperature data

(76) Inventors: Francesco Pompei, Cambridge, MA (US); Janette H. Lee, Everett, MA (US); Jason N. Jarboe, Somerville, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/728,956

(22) Filed: Mar. 27, 2007

(51) Int. Cl. G11B 7/00; A61B 5/00

(52) U.S. Cl. 369/100; 600/474

(57) ABSTRACT

An identification input device and a temperature detector that detects body temperature data. The identification input device may be an optical touch pen. The infrared touch pen may be affixed to the temperature detector using a hook and connector or a snap fit connector. In use, the temperature detector computes body temperature data and the identification input device determines an identifier. The identifier is unique to a user. After obtaining the body temperature data and identifier, a transmitter may transmit body temperature data and the identifier over a wireless communications path to a processing unit. By transmitting data over a wireless communications path, an individual's data or a large group of data may be analyzed and viewed via a display unit.

### BACKGROUND OF THE INVENTION

In recent years, infrared thermometers have come into wide use for detection of temperature. One such infrared thermometer presented in U.S. Pat. No. 6,292,685 (incorporated by referenced in its entirety) is depicted in FIG. 1. In particular, FIG. 1 illustrates the temporal arteries 12 and 14 that extend upwardly toward the side of the human face and bifurcate at 16 and 18 in the forehead region. In that region, the temporal artery passes over the skull bone very close to the skin and is thus termed the superficial temporal artery. The superficial temporal artery is, therefore, particularly accessible for providing temperature readings and, as an artery, has a temperature close to the heart temperature. Further, there are no known arterial/venus anastomoses, that is, shunts between the artery and veins for regulation of skin temperature. Accordingly, the blood flow is relatively stable, varying a maximum of only 50% as opposed to as much as 500% in other areas of the skin.

To locate the temporal artery, a temperature sensor, preferably a radiation detector 20, is scanned across the side of the forehead over the temporal artery while electronics in the detector search for the peak reading which indicates the temporal artery. Preferably, that temperature reading is then further processed in accordance with an algorithm specific to the temporal artery for providing a display temperature which may, for example, correspond to core, oral or rectal temperature.

Thermometers, such as the one described above, may be used to obtain a large number of temperature readings in a short period of time. For example, runners at a marathon may have their temperatures read at the end of a race. Likewise, in the case of a disease outbreak temperature readings of many people may be taken in a screening process.

### SUMMARY OF THE INVENTION

Typically, a person manually obtains a body temperature, associates the data to an individual, and stores the data (e.g., in a log book). The larger the group of people, the larger the list of individuals and corresponding data. For example, marathon runners typically have bibs having identifying numbers. The body temperature data of each runner is detected using a preferred temperature detector, such as a temporal artery thermometer using an arterial heat balance approach, and association with the individual's number. After collecting the body temperature data in a log, the body temperature data is input into a processor, such as a laptop, for processing.

In an example embodiment of the present invention, an identifier of an individual is determined and the individual's temperature is taken with a temperature detector. The individual's body temperature data and identifier are transmitted over a wireless communications path to a processor. By transmitting data over a wireless communications path, a large group of data may be collected and analyzed efficiently.

An individual's unique identifier may be determined by an identification reader, such as a laser diode based scanner, LED contact scanner, optical scanner, magnetic scanner, a bar code reader, a magnetic strip reader, character reader, or keypad input device. The body temperature data and identifier are stored in memory of a processor. The temperature may be displayed to a user on a thermometer, a computer or both. The display may for example, be of individual data or be a histogram of computed data. It is useful to note that the thermometer and computer may be operated by one or multiple users (e.g., one user taking the temperature and another user using the laptop computer).

In one convenient implementation, the temperature detector and identification reader are both compiled through respective electronic cables to a wireless transmission. For further convenience the reader and detector may be joined by a hook and loop connector such as VELCRO™. Similarly, other connectors, such as a snap fit connector, may be used to join the reader and detector.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates an infrared thermometer scanning the temporal artery.

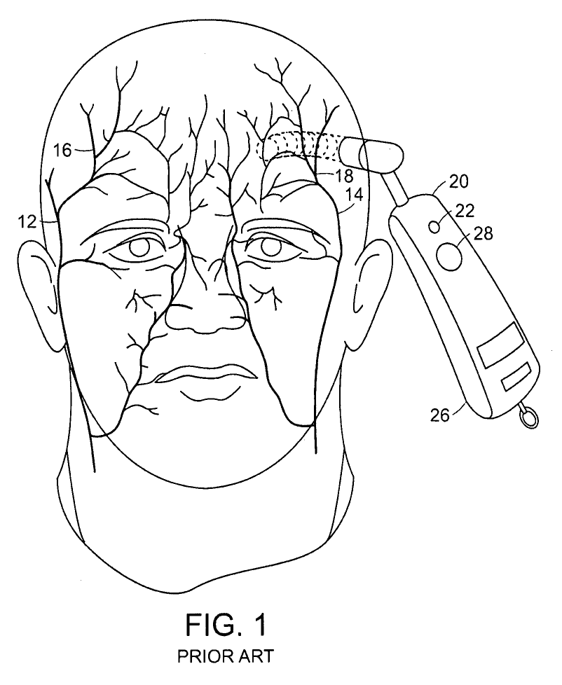

FIG. 2A is a high level view of an collecting and transmitting temperature data.

FIG. 2B is a detailed view of a unit for collecting temperature data and an identifier.

FIG. 3 is a flow diagram illustrating an collecting body temperature data.

### DETAILED DESCRIPTION OF THE INVENTION

A description of example embodiments of the invention follows.

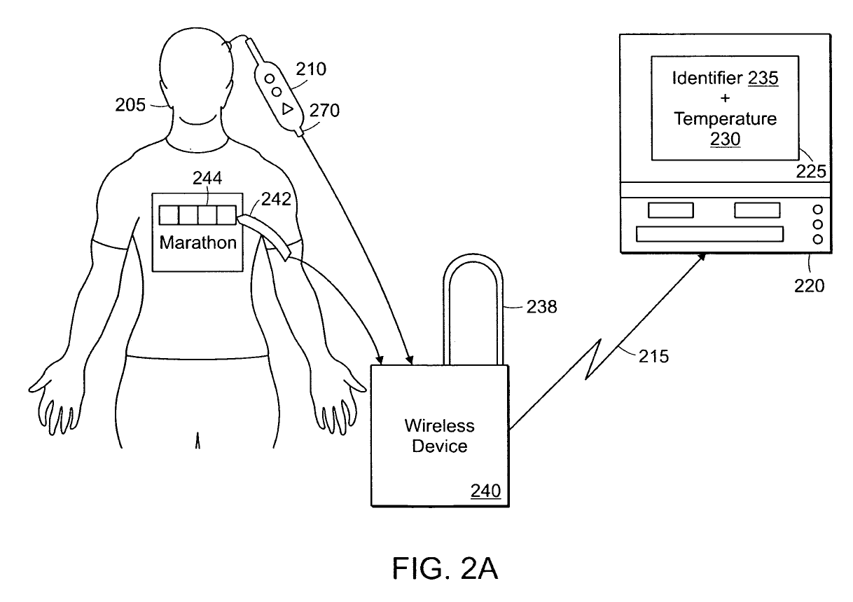

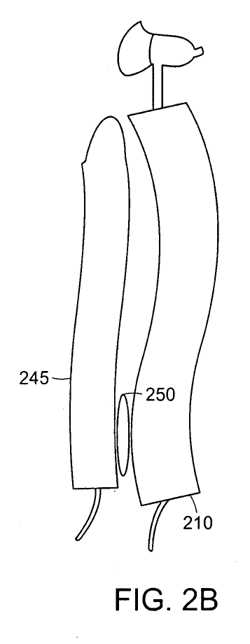

FIG. 2A is a high level view of collecting and transmitting temperature data that includes a body portion 205, a temperature detector 210, a wireless communications path 215, a wireless device 240, a strap, a touch pen 242, an identification source 244, a processor 220, and processor display 225. In use, a user swipes a touch pen 242, which communicates an identifier 260 from a identification source 244 to the wireless device 240. For example, an identification source 244 may be a corporate pass, employee identification, or government issued identification having an unique identification information (e.g., an identifier 260). In particular embodiments, an identification source 244 may be a bib of a marathon runner having a bar code (e.g., an identifier 260), which is unique to each runner. The barcode signal or a corresponding identifier 260 is communicated to the wireless device 240. The temperature detector 210 detects body temperature data and sends the temperature to the wireless device 240. In particular embodiments, the temperature detector 210 uses an RS-232 output (e.g., a communication port 270). The RS-232 output transmits temperature to the wireless device 240. After receiving the temperature and identifier 260, the wireless device 240 transmits the identifier 260 and temperature to a processor 220 using a wireless connection.

In this example embodiment, the temperature detector 210 obtains a temperature reading from a body portion 205. For example, an internal core temperature can be computed using an arterial heat balance. The teachings of calculating body temperature data is described in U.S. Pat. No. 6,292,685, which is hereby incorporated by referenced. It is useful to note embodiments of the present invention are not limited to temporal artery readings. Instead, any type of temperature detector may be used, including axillary, ear, or non radiation detectors. Moreover, the processor 220, instead of a thermometer, may also calculate the temperature reading upon receiving the raw temperature data such as heat flux and ambient temperature data.

After obtaining the temperature, the temperature is sent to a wireless device 240. The wireless device 240 obtains an identifier 235 using, for example a keypad or reader (not shown). In an convenient embodiment, the wireless device 240 uses a strap 238 that allows for portability of the device. The wireless device 240 transmits the temperature data and the identifier 235 over a communications path 215 to a processor 220. Upon receiving the temperature data and the identifier 235, the processor 220 may associate a time and/or date to the data. The processor 220 may be a laptop, desktop, portable device, or other device used to receive, process, and analyze data. The processor may display the temperature reading and the identifier 235 on the processor display 225. In operation, a user (not shown) uses the processor display 225 for reviewing the data of one or more temperature detectors. For example multiple temperature detectors (as many as the communications protocol allows) may be transmitting data to a computer over a wireless communications path. By transmitting data over a wireless communications path, a large group of data may be collected and analyzed from one or more detector units. One advantage of measuring a large group of data and using a screening process, for example, is a user may predict an outbreak in a geographic area. For example, a histogram may be obtained for a location allowing for early detection of an outbreak. It is useful to note that the data may also be processed before transmission to the processor.

The processor 220 receives the temperature and the identifier 235 via a wireless communications path 215. Upon receiving the temperature and the identifier 235, the processor 220 begins a software application (not shown) to display the data. The software application displays the temperature and the identifier 235 data in the processor display 225. The processor display 225 may, for example, include a temperature and a barcode reading for one or more readings of each of many individuals including histogram data, a warning if temperature is above the user-specified high-temperature limit, a history of temperature and time for a particular individual, an aggregate data collection, or other relevant data.

In particular, data may be used to evaluate screening programs at airports, schools, factories and other populated environments, during perceived potential epidemics for persons who may be at risk for transmitting epidemic diseases. Such evaluations may be aided by the analysis of the data statistically to identify persons with unexpectedly high temperatures, indicating possible fever. Such identified persons would be detained briefly for closer examination by a medically trained person. The data would allow use of mathematical optimization routines which would maximize the sensitivity for detecting sick individuals to prevent the spread of disease, while minimizing false positives, which unnecessarily inconveniences people and adds delay.

In particular embodiments, data may be used to alert medical professionals of high or low temperatures. More specifically, aggregate data may be used for early detection of outbreaks where high temperature is an indicator. For example, aggregate data may be used to determine how marathon runners or other population behave physiologically with respect to weather, distance run, time, or some other factor.

In the case of marathons, runner bibs typically have a human readable number and a corresponding mach readable bar code. The bar code is scanned and the resulting signals are decoded into the ASCII characters, which were originally encoded into the bar code. Barcodes can be read by optical scanners called barcode readers or scanned from an image by special software. In alternative embodiments, a laser diode based scanner, LED contact scanner, optical or magnetic scanner, character reader, Radio Frequency Identification (RFID), or other suitable reader may be used. In particular embodiments, a reader includes a transmission device to interact directly with a processor.

FIG. 2B is a view of a unit for collecting temperature data and an identifier. A temperature detector 210 attaches to a touch pen 245 using a suitable adhesive material 250, such as a hook and loop connector (e.g., VELCRO™) or a snap fit connector. In operation, a user (not shown) separates a touch pen 245 from a temperature detector 210. The user swipes the touch pen 245, which communicates an individual's identification (e.g., an identifier) to a transmitter (not shown). Similarly, the temperature detector 210 detects body temperature data and sends temperature data to a transmitter as described above. In an convenient embodiment, a temperature detector includes a barcode reader and wireless transmitter. Thus, temperature data and an individual's identification may be determined and transmitted from a single unit.

The transmitter establishes a wireless connection using BLUETOOTH™, infrared or other suitable communication protocol. BLUETOOTH™ is an industrial specification for wireless Personal Area Networks (PANs). BLUETOOTH™ provides a way to connect and exchange information between devices such as mobile phones, laptops, PCs, printers, digital cameras, and video game consoles over a secure, globally unlicensed short-range radio frequency. The BLUETOOTH™ specifications are developed and licensed by the BLUETOOTH™ Special Interest Group.

In particular embodiments, the corresponding apparatus includes a temporal artery thermometer, a barcode reader, a BLUETOOTH™ transmitter and a BLUETOOTH™ enabled laptop. The thermometer and a barcode reader are connected by retractile cords to an enclosure for battery power and a BLUETOOTH™ radio transmitter. The temperature and barcode data is sent via a wireless serial connection, such as a BLUETOOTH™ protocol, to a laptop, where the data is displayed and collected.

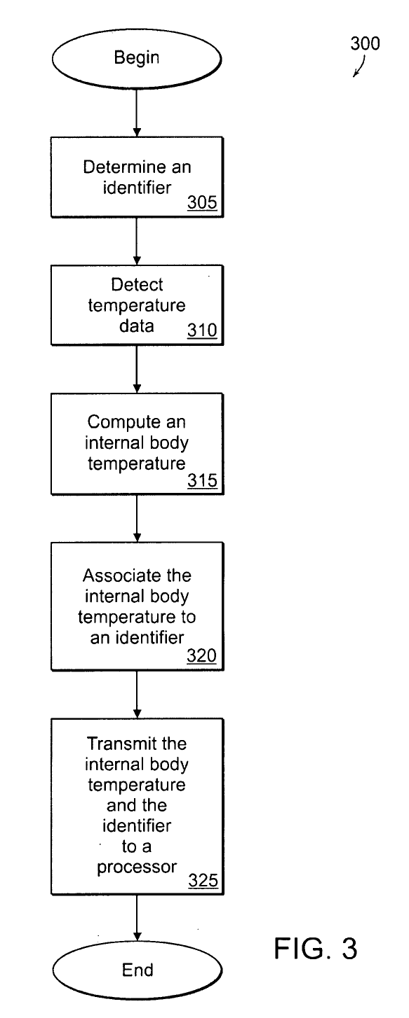

FIG. 3 is a flow diagram 300 illustrating an example temperature reading and transmission process. After beginning, the process determines (305) an identifier for a user. Next, the process detects (310) temperature data. For example, an embodiment detects temperature at a forehead through the lateral scan across the temporal artery. After detecting the temperature, the process computes (315) an internal body temperature of the body. It is useful to note step 315 may also be done later on the processor. Next, the process associates (320) the internal body temperature to the identifier. Once the internal body temperature has an identifier, the process transmits (325) the internal body temperature and the identifier over a wireless communications path to a processor.

It should be understood that any of the processes disclosed herein, such as transmitting body temperature data, or the flow diagram of FIG. 3, may be implemented in the form of hardware, firmware, or software. If implemented in software, the software may be processor instructions in any suitable software language and stored on any form of computer readable medium. The processor instructions are loaded and executed by a processor, such as a general purpose or application specific processor, that, in turn, performs the example embodiments disclosed herein.

While this invention has been particularly shown and described with references to example embodiments thereof, it will be understood by those skilled in the art that various changes in form and details may be made therein without departing from the scope of the invention encompassed by the appended claims.

What is claimed is:

1. A method of collecting temperature data, comprising:
determining an identifier of an individual for each of plural individuals;
detecting body temperature data of the individual associated with the identifier; and
transmitting the body temperature data and the identifier over a wireless communications path to a processor.

2. The method of claim 1 wherein determining an identifier of the individual is through an identification reader.

3. The method of claim 2 wherein the identification reader is a touch pen.

4. The method of claim 3 wherein the touch pen is a bar code reader.

5. The method of claim 3 wherein the touch pen is affixed to a temperature detector using a connector.

6. The method of claim 2 wherein the identification reader includes a bar code reader.

7. The method of claim 1 wherein detecting body temperature data of the individual further comprises scanning across the temporal artery to obtain body temperature data.

8. The method of claim 1 further comprising storing the body temperature data and the identifier in memory of the processor.

9. The method of claim 1 further comprising processing the body temperature data using the processor.

10. The method of claim 9 further comprising displaying a result for the body temperature data to a user of the processor.

11. An apparatus for collecting a temperature data, comprising:
an identification input device;
a temperature detector; and
a transmitter that transmits body temperature data from the temperature detector and an identifier from the identification input device over a wireless communications path to a processing unit.

12. The apparatus of claim 11 wherein the identification input device is an identification reader.

13. The apparatus of claim 12 wherein the identification input device is a touch pen.

14. The apparatus of claim 13 wherein the touch pen is a bar code reader.

15. The apparatus of claim 13 wherein the touch pen is affixed to the temperature detector by using a connector.

16. The apparatus of claim 11 wherein the identification input device includes a bar code reader.

17. The method of claim 11 wherein the temperature detector scans across the temporal artery to obtain body temperature data.

18. The apparatus of claim 11 further comprising memory in the processor to store the body temperature data and the identifier.

19. The apparatus of claim 11 further comprising the processing unit processes the body temperature data.

20. The apparatus of claim 19 further comprising a display unit of the processor that displays a result for the body temperature data to a user.

21. An apparatus for collecting a temperature data, comprising:
means for determining an identifier of an individual for each of plural individuals;
means for detecting body temperature data of the individual associated with the identifier; and
means for transmitting the body temperature data and the identifier over a wireless communications path to a processor.
